(19) United States
(12) Patent Application Publication
(10) Pub. No.: US 2014/0276917 A1
(43) Pub. Date: Sep. 18, 2014

(54) Kit for clamping an umbilical cord safely and for preventing misidentification and unauthorized removal of a newborn

(71) Applicant: Dean Trivisani, Oakland, NJ (US)

(72) Inventor: Dean Trivisani, Oakland, NJ (US)

(21) Appl. No.: 14/202,872

(22) Filed: Mar. 10, 2014

Related U.S. Application Data
(60) Provisional application No. 61/786,834, filed on Mar. 15, 2013.
(60) Provisional application No. 61/816,274, filed on Apr. 26, 2013.

(51) Int. Cl. A61B 19/00; A61B 17/122

(52) U.S. Cl. CPC A61B 19/44; A61B 17/122
USPC 606/120

(57) ABSTRACT

The present disclosure relates to a kit comprising numbered and unnumbered umbilical cord clamps and matching numbered wristbands. The unnumbered clamps are for initial clamping of the umbilical cord immediately after the birth and are distinguished from the numbered clamps by size, shape and/or by color. The unnumbered clamps are most preferably of gender neutral color, such as yellow. The numbered clamps may be white or may be of color indicating the newborn's gender. The numbered clamps are used to reclamp the umbilical cord below the unnumbered clamp after the newborn is cleaned. The unnumbered clamp is cut off and disposed once the numbered clamp is in its place. The wristbands have the same numbers as the numbered clamps. Two wristbands are attached to the newborn, one wristband is for the mother and one wristband may be for the significant other. The newborn may not be removed from a defined area without the numbers in the wristbands and on the clamp matching. The clamp and the wristbands may have additional security codes. An alarm may be triggered if the numbers in the clamp and the wristband are not matching when the newborn is removed from the defined area.

### PRIORITY CLAIM

This application claims priority of the U.S. provisional patent application No. 61/786,834 filed on Mar. 15, 2013 and 61/816,274 filed on Apr. 26, 2013. The contents of the priority applications are incorporated herein by reference in their entirety.

### FIELD OF THE INVENTION

The invention relates to safe identification of newborn. Especially the method relates to vascular clamps and means to safely clamp the umbilical cord and to lower incidents of misidentifying newborns and to safely identify newborns. The invention also relates to systems which are useful to hospitals with newborns to be protected from unauthorized removal.

### BACKGROUND OF THE INVENTION

General hospital-based obstetric practice introduces artificial clamping as early as 1 minute after the birth of the child. It is common that the doctor places the clamp in a hurry and as a result the clamp may be located distantly from the base of the umbilical cord. On these occasions the baby has to be reclamped. This is usually done later by a nurse. A common practice today is to use umbilical clamps that are numbered or otherwise marked and paired with an identifier for the mother having the same numbering or marking. The umbilical clamps lock to the cord in a way that they cannot be removed without breaking them. Thus, when the nurse needs to reclamp the child it means that a new matching clamp for the child and additional identifier for the child and mother has to be provided. Thus the currently used reclamping process is wasteful and inefficient. In addition to increased costs, the currently used system increases chances of mismatching the baby and the mother, because the nurse may forget to provide a new identifier to the child (numbered wrist band) and mother when reclamping the baby.

There have been attempts to solve the above described problems but none of the available devices or methods addresses all of the problems. The invention described in this application provides a solution to the current inefficient practice.

U.S. Patent Application No. 2004099178 aims to solve a problem of poorly attached clamps by providing a releasably locking umbilical cord clamp. The clamp according to this disclosure however is rather large. The clamp includes one or more of: a gender-identifying color, a grasping portion to facilitate closing and locking the clamp on the remaining umbilical cord, a fluid removal channel, a separable measurement portion to facilitate positioning of the clamp at a suitable location on an umbilical cord, and an operatively associated key to unlock and reposition or remove the clamp. Also included are methods relating to using a gender-identifying color on an umbilical cord clamp to facilitate gender and identity recognition and methods of applying the clamp at the proper distance.

U.S. Pat. No. 5,006,830 attempts to solve the problem of removing a new born from a defined area without permission. The patent discloses a locking umbilical cord clamp with an attached identification mark and an attached triggering device capable of triggering a detection system upon removal of the umbilical clamp from the defined area and a wristband with an identification mark thereof corresponding to the identification mark on the umbilical clamp for attachment to the wrist of a person authorized to remove the newborn from a defined area. The flaw in this system, however is that when the doctor initially attaches the numbered clamp on the umbilical cord it either has to have an unlocking mechanism to be removed in case it needs to be attached on different position, or it needs to be broken and a new clamp is to be attached. As the new clamp would have a different number, the wrist bands have to be thrown away also and a new set of matching wristbands and clamp need to be taken in use.

Similarly, U.S. Pat. No. 5,608,382 attempts to solve the problem of removing a new born from defined area without authorization. The patent discloses an infant identification and security system including an umbilical cord clamp and a matching wristband for the infant's mother, with a pair of information storage modules attached to the clamp and wristband and a compatible terminal for reading and writing information thereto. Triggering elements are included for triggering a compatible alarm system if an unauthorized person removes the newborn from the maternity ward. This patent poses the same problem as described above.

Another attempt to prevent unauthorized removal of the newborn is provided in U.S. Pat. No. 5,512,879. This patent discloses a miniature electronic security tag affixed to the ankle of newborn infants that contains an RF-transmitter and a digital encoding circuit. The tag permits continuous monitoring to alert a central monitoring computer if the tag is cut or stretched or if an unauthorized person attempts to leave the hospital with the infant.

An attempt to solve problem of matching the child and the mother is provided in U.S. Pat. No. 6,212,808. The patent discloses a safety identification assembly for use in neonatology including identifying sub-assemblies detachably connected to each other for identifying mother and baby and for closing umbilical cord ends.

As is clear from above, there are a number of various designs for umbilical clamps but there is nothing that would address the problem described above. This invention solves the problem.

### SUMMARY OF THE INVENTION

The invention relates to a kit of umbilical cord clamps comprising at least one unnumbered clamp preferably with a specific gender neutral color, such as yellow, and at least one numbered clamp with a set of matching numbered wrist bands. The color yellow is also widely used to denote disposable/single use items. When the baby is born, an unnumbered, gender neutrally colored clamp is handed to the doctor and he/she clamps the umbilical cord immediately. Later when the newborn is cleaned and stabilized the nurse will reclamp the cord at an appropriate distance from the cord base with one of the numbered clamps, and remove and dispose the yellow unnumbered clamp. The clamp should be applied parallel to the abdomen leaving approximately ½ inch between the clamp and abdomen and ½ to ¾ of an inch above the clamp to allow for shrinkage of the cord. The numbered clamps have a different color than the unnumbered clamp, generally it may be white or the color may be for example blue or pink to indicate the gender. The kit comprises also a set of matching numbered wrist bands; preferably the kit comprises at least three, and preferably four wrist bands; two for the baby, one for the mother and if required, one for the significant other. The clamps are preferably also compatible with or capable to accommodate RFID (radio frequency identification) umbilical type transponders as an additional security. The wristbands may also be colored with a color indicating the baby's gender. One of the two wristbands for the baby is attached to the baby's arm and another to baby's ankle. The numbering on the clamps and on the wristbands may be a barcode and the barcodes would be read and checked to assure that the barcodes on the wrist bands of the mother or significant other and the barcodes on the clamps and baby's bands match before the baby can be removed from a defined area. The numbered clamp may also include an alarm system activated when a person with unmatching wristband tries to remove the baby from the area.

It is an object of this invention to provide a kit that allows safe clamping and reclamping of the umbilical cord of a newborn.

Another object of this invention is to provide a kit allowing positive identification of the baby and ability to match the mother and a significant other with the newborn.

It is yet another object of this invention to provide a system to prevent unauthorized removal of a newborn from a confined area.

It is an object of this invention to provide a kit for safe clamping of an umbilical cord and identification of a newborn, said kit comprising: at least one unnumbered umbilical cord clamp with a locking mechanism to be used for clamping umbilical cord of a newborn immediately after birth; at least one numbered umbilical cord clamp with a locking mechanism to be used for reclamping the umbilical cord, wherein the unnumbered and numbered clams are distinguishable from each other by color, shape, size or a combination thereof; and at least three identically numbered wristbands, at least two of which are for the newborn and at least one for authorized adults, where numbering of the wristbands matches with numbering of the numbered clamp.

It is a further object of this invention to provide a method to prevent unauthorized removal of a newborn, said method comprising the steps of: a) providing a kit comprising a umbilical cord clamp with a specific number and at least three wristbands having the same specific number; b) clamping newborn's umbilical cord with the clamp of step a); c) attaching one wristband on newborn's ankle and one wristband on newborn's wrist; d) providing one wristband to each authorized adult; e) checking that the numbers of the clamp and all wristbands match with each other when a newborn is removed from a defined area; and wherein an alarm is triggered when anyone of the numbers in step e) does not match.

### BRIEF DESCRIPTION OF THE DRAWINGS

FIG. 1 shows a kit with one unnumbered clamp, one numbered clamp and four wristbands matching with the numbered clamp.

FIG. 2 shows a new born whose umbilical cord has been clamped with an unnumbered clamp. The clamping has been done immediately after the birth and the clamp locates relatively far away from the base of the cord.

FIG. 3 shows a new born whose umbilical cord has been reclamped with a numbered clamp. Now the numbered clamp is close to the base of the cord. The unnumbered clamp attached further away from the base of the umbilical cord base is removed by cutting the cord between the numbered clamp and unnumbered clamp.

FIG. 4 shows the umbilical cord clamped with a numbered clamp after the unnumbered clamp has been removed by cutting the umbilical cord from between the numbered and unnumbered clamp. The numbered clamp is shown to have an RFID (radio frequency identification) umbilical type transponders attached therein.

### DESCRIPTION OF THE PREFERRED EMBODIMENTS

The preferred embodiments of the present invention will now be described with reference to FIGS. 1 to 4.

Referring now to FIG. 1, the kit according to this invention comprises at least one unnumbered umbilical cord clamp 10, at least one numbered umbilical cord clamp 20 with identification numbers 25, at least two numbered wristbands 30 for the baby where the identification numbers 35 match with identification numbers of the clamp, at least two numbered wristbands 40 for the authorized adults where the identification numbers 45 match with the identification number of the clamp and with the identification numbers of the two wristbands of the baby. The kit may also include a radio frequency identification umbilical type transponders 50 to be attached in the numbered clamp (shown in FIG. 4).

The clamps according to this invention have a locking mechanism to secure the clamp on the cord. The clamps are preferably as described and shown in U.S. patent application Ser. No. 13/117,192 and U.S. Pat. No. D673,272, both of which are incorporated herein by reference. The unnumbered clamp 10 is preferably of a different color than the numbered clamps and is preferably of a gender neutral color. Most preferably the gender neutral color is yellow, which is often used to indicate disposable nature of an item. This would enable the doctor to pick the unnumbered clamp even when he is in a hurry. However, other means of identifying the neutral clamp could also be utilized, such as an exaggerated size or shape of the clamp. The kit may contain more than one unnumbered gender neutrally colored clamps in case the clamps slip from the hands of the doctor.

Still referring to FIG. 1, two of the wristbands 30 are for the baby to be attached to an ankle and a wrist of the baby. The numbering 35 of the bands matches the numbering 25 of the numbered clamp 20. The other two wristbands 40 are for the mother and the significant other. The numbering 45 on these wristbands 40 matches the numbering 35 of the wristbands of the baby 30 and of the numbering 25 of the numbered clamp 20.

Referring now to FIG. 2, the unnumbered umbilical cord clamp 10 has been attached to the umbilical cord 1 of the newborn. It often happens that immediately after the birth the doctor is in an extreme hurry to clamp the umbilical cord, or the doctor determines there is a need for a procedures involving the umbilical cord, and therefore the clamp is most often attached at a location not very close to the base of the cord. This happens because it is extremely important to get the clamp on the cord quickly especially if the baby is in distress and there may not be time to perfect the attachment. Furthermore, this clamping is conducted before the baby is washed and body fluids make the cord and doctor's hands slippery, resulting in an imperfect clamping. The clamp may be applied incorrectly because the baby may be in distress and the baby needs to be rushed to NICU (neonatal intensive care unit). Even in non emergency situation there may be amniotic fluid or meconium on the cord and therefore perfect clamping may not be possible due to slippage. However, it is important to clamp the umbilical cord properly at a proper length and horizontally to the baby's abdomen in order to prevent cord pull and irritation at the base of the cord stump. If applied at an improper angle the clamp may also put additional pressure on one side of the cord thereby tearing the cord and causing the baby to bleed out. According to this invention, the purpose of the first clamping with the unnumbered preferably yellow clamp is simply to close the end of the umbilical cord to avoid infections and other problems. Furthermore, when the cord is clamped too far from the base of the cord the child's belly button will have an unaesthetic appearance. For these reasons a later reclamping is usually needed after the baby is cleaned and washed.

The unnumbered clamp has to be marked in a way that the doctor in a hurry can distinguish it from the numbered clamps. One preferred way to distinguish the unnumbered clamp from the numbered ones is to distinguish by color: the unnumbered clamp is of different color than the numbered ones. Most preferably the unnumbered clamp is yellow. With a quick glance the doctor would be able to pick the unnumbered clamp without any detailed inspection of the numbering/non numbering. Other preferable means to distinguish the unnumbered clamp would be size of the clamp. The unnumbered clamp may be clearly bigger than the numbered clamps. That would also enable the doctor to quickly pick the unnumbered clamp without any extra inspections of the numbering. Yet another way to distinguish the unnumbered clamp would be different shape of the clamp. However, the color yellow is the most preferred embodiment because yellow is generally used as an indication of disposable items. Therefore, the color yellow would be a direct indication to any doctor that the clamp is unnumbered temporary clamp.

Referring now to FIG. 3, the numbered umbilical cord clamp 20 has been attached to the umbilical cord 1 of the newborn. The numbered clamp 20 has been attached close to the base of the umbilical cord. The numbered clamp has been attached after the baby has been washed and cleaned and a nurse has the required time to attach the numbered clamp close to the base of the umbilical cord's base to ensure that the belly button will eventually be symmetric and aesthetically acceptable and that no additional pressure or tearing is put by the clamp. The original unnumbered clamp 10 is shown still to be attached on the cord and it is to be removed by cutting the cord from between the numbered and unnumbered clamp. The numbered clamp is of different color than the unnumbered clamp. The numbered clamp may be white, or it may be blue or pink according to the gender of the child.

According to one preferred embodiment, the numbering in the clamp and on the three, preferably, four wristbands are in the form of a pin code or a bar code. According to one preferred embodiment additional ID recognition is included in the numbered clamps and the wristbands. Such additional ID recognition may include additional pin code or bar code tags separately attached to the clamp and matching pin or bar codes are attached to the wristbands. When someone tries to remove the baby from the defined area, the numbering and/or the codes of the clamp as well as the wristbands are checked to verify that they match with each other. Without matching numbers and codes in all wristbands and the numbered clamp, the baby cannot be removed from the area. If the clamp and the wristbands are additionally colored, the colors need to also match.

Referring to FIG. 4: The unnumbered clamp shown in FIG. 3 has been removed by cutting the umbilical cord between the numbered an unnumbered clamps. According to one preferred embodiment the additional ID recognition may include RFID (radio frequency ID) tags or chips. In this figure a radio transponder 50 is attached into the numbered clamp 20. According to one preferred embodiment the numbered clamps and the wristbands may include an alarm system such that if a person with mismatching wristband numbering tries to remove the baby from the defined area an alarm will be triggered. The alarm system may be a RTLS (real time location system) including RFID-tags or chips. The alarm can be a sound signal or a light signal or it can be a signal preventing an exit door to open.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A kit for safe clamping of an umbilical cord and identification of a newborn, said kit comprising:
at least one unnumbered umbilical cord clamp with a locking mechanism to be used for clamping umbilical cord of the newborn immediately after birth;
at least one numbered umbilical cord clamp with a locking mechanism to be used for reclamping the umbilical cord, wherein the unnumbered and numbered clamps are distinguishable from each other by color, shape, size, or a combination thereof; and
at least three identically numbered wristbands, at least two of which are for the newborn and at least one for an authorized adult, where numbering of the wristbands matches with numbering of the numbered clamp.

2. The kit of claim 1, wherein the numbered clamp and/or the wristband has an additional ID recognition.

3. The kit of claim 2, wherein the additional ID recognition is a bar code tag.

4. The kit of claim 2, wherein the additional ID recognition includes a real time location system (RTLS).

5. The kit of claim 4, wherein the RTLS includes radio frequency ID (RFID) chips or tags.

6. The kit of claim 1, wherein the unnumbered clamps are distinguished from the numbered clamps by the color.

7. The kit of claim 1, wherein there are four wristbands.

8. The kit of claim 2, wherein the kit comprises two unnumbered umbilical cord clamps, two numbered umbilical cord clamps, and four wristbands.

9. The kit of claim 6, wherein the unnumbered umbilical cord clamps are of gender neutral color.

10. The kit of claim 9, wherein the unnumbered umbilical cord clamps are yellow.

11. The kit of claim 1, wherein the kit additionally comprises a radio transponder attachable to the numbered clamp.

12. A kit for safe clamping of an umbilical cord and identification of a newborn, said kit comprising:
at least one unnumbered gender neutrally colored umbilical cord clamp with a locking mechanism to be used for clamping umbilical cord of the newborn immediately after birth;
at least one numbered umbilical cord clamp with a locking mechanism to be used for reclamping the umbilical cord, said clamp having a color different from the color of the unnumbered clamp; and
at least three identically numbered wristbands, at least two of which are for the newborn and at least one for an authorized adult, where numbering of the wristbands matches with numbering of the numbered clamp.

13. The kit of claim 12, wherein the kit has two yellow unnumbered clamps and two numbered clamps of other than yellow color.

14. A method to prevent unauthorized removal of a newborn, said method comprising the steps of:
a. providing a kit comprising a umbilical cord clamp with a specific number and at least three wristbands having the same specific number;
b. clamping newborn's umbilical cord with the clamp of step a),
c. Attaching one wristband on newborn's ankle and one wristband on newborn's wrist;
d. Providing one wristband to each authorized adult;
e. checking that the numbers of the clamp and all wristbands match with each other when a newborn is removed from a defined area; and
wherein an alarm is triggered when anyone of the numbers in step e) does not match.

15. The method of claim 14, where the clamp and the wristbands have an additional ID recognition.

16. The method of claim 14, wherein the additional ID recognition is a bar code.

17. The method of claim 14, wherein the additional ID recognition includes a real time location system (RTLS).

18. The method of claim 17, wherein the RTLS includes radio frequency ID-chips or tags.

19. The method of claim 16, wherein the matching of the bar codes is checked in step e).

20. The method of claim 14, the alarm prevents opening of an exit.
